(19) United States
(12) Patent Application Publication
(10) Pub. No.: US 2010/0081845 A1
(43) Pub. Date: Apr. 1, 2010

(54) Process for Production of Optically Active Benzylamine Derivatives

(75) Inventors: Teruaki Sugiyama, Hida-shi (JP); Akinori Fujishima, Hida-shi (JP); Atsushi Umetani, Hida-shi (JP); Nobutaka Matsunaga, Hida-shi (JP); Yoshiyuki Omori, Hida-shi (JP); Norihiko Seko, Hida-shi (JP)

Correspondence Address: CROMPTON, SEAGER & TUFTE, LLC, 1221 NICOLLET AVENUE, SUITE 800, MINNEAPOLIS, MN, 55403-2420, US

(73) Assignee: ALPS PHARMACEUTICAL INC. CO., LTD., Hida-shi, Gifu-ken (JP)

(21) Appl. No.: 11/993,821

(22) PCT Filed: Jun. 20, 2006
(86) PCT No.: PCT/JP2006/312367

§ 371 Date: Dec. 21, 2007

(30) Foreign Application Priority Data
Jun. 27, 2005 (JP) 2005-187347

(51) Int. Cl. C07C 221/00

(52) U.S. Cl. 564/343

(57) ABSTRACT

A benzylamine derivative has a structure represented by the following formula (1):

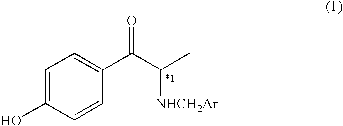

In a method for optical resolution of the benzylamine derivative, optically active mandelic acid is used as an optical resolving agent. In an optical resolution step in a production method of the optically active benzylamine derivative, an optically active (S)-benzylamine derivative represented by the formula (3) is precipitated as (S)-mandelic acid salt from a solution containing the benzylamine derivative and (S)-mandelic acid:

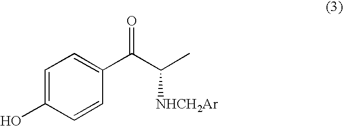

wherein Ar represents an aryl group that has 6 to 15 carbon atoms and may have a substituent, and *1 represents an asymmetric carbon atom.

### TECHNICAL FIELD

The present invention relates to a novel benzylamine derivative useful as a pharmaceutical intermediate, a method for optical resolution of the benzylamine derivative, and a process for production thereof. Further, the present invention relates to a process for production of an optically active benzylamine derivative or (1R,2S)-2-amino-1-(4-hydroxyphenyl)propan-1-ol from a benzylamine derivative.

### BACKGROUND ART

Conventionally, 1-(4-benzyloxyphenyl)-2-dibenzylamino-1-propanol has been known as a benzylamine derivative, and this benzylamine derivative has been disclosed as a synthetic intermediate of (1R,2S)-2-amino-1-(4-hydroxyphenyl)propan-1-ol, which is an optically active substance (for example, see Non-Patent Document 1).
[Non-Patent Document 1] Journal of Medicinal Chemistry, 1977, vol. 20, No. 7, 978-981

### DISCLOSURE OF THE INVENTION

An objective of the present invention is to provide a benzylamine derivative significantly useful for production of an optically active benzylamine derivative and a process for production thereof, and a method for optical resolution of a benzylamine derivative, by which an optically active benzylamine derivative having a specific structure is easily obtained from the benzylamine derivative, a process for production of an optically active benzylamine derivative, and a process for production of (1R,2S)-2-amino-1-(4-hydroxyphenyl)propan-1-ol.

One aspect of the present invention provides a benzylamine derivative having a structure represented by the following formula (1):

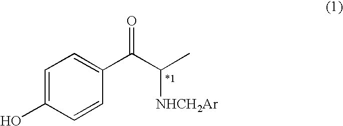

wherein Ar represents an aryl group that has 6 to 15 carbon atoms and may have a substituent, and *1 represents an asymmetric carbon atom.

Another aspect of the present invention provides a benzylamine derivative having a structure represented by the following formula (2):

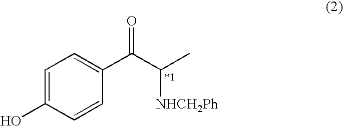

wherein Ph represents a phenyl group, and *1 represents an asymmetric carbon atom.

Yet another aspect of the present invention provides a method for optical resolution of each of the benzylamine derivatives described above, wherein an optically active mandelic acid is used as an optical resolving agent.

Yet another aspect of the present invention provides a process for production of a benzylamine derivative having a structure represented by the above formula (1), including a step of performing a substitution reaction of 2-bromo-(4-hydroxyphenyl)propan-1-one to obtain a benzylamine derivative.

Yet another aspect of the present invention provides a process for production of an optically active benzylamine derivative, wherein an optically active (S)-benzylamine derivative having a structure represented by the following formula (3) is produced from the benzylamine derivative having a structure represented by the above formula (1), including a step of precipitating (S)-mandelic acid salt of the optically active (S)-benzylamine derivative from a solution containing the benzylamine derivative and (S)-mandelic acid as an optical resolving agent to optically resolve the benzylamine derivative,

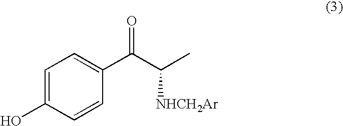

wherein Ar represents an aryl group that has 6 to 15 carbon atoms and may have a substituent.

Yet another aspect of the present invention provides a process for production of an optically active benzylamine derivative, wherein an optically active (S)-benzylamine derivative having a structure represented by the above formula (3) is produced from a benzylamine derivative having a structure represented by the above formula (1), including a step of precipitating (R)-mandelic acid salt of the optically active (R)-benzylamine derivative having a structure represented by the following formula (4) from a solution containing the benzylamine derivative and (R)-mandelic acid as an optical resolving agent to optically resolve the benzylamine derivative,

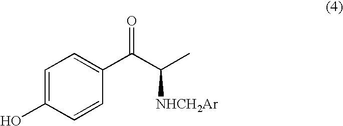

wherein Ar represents an aryl group that has 6 to 15 carbon atoms and may have a substituent.

These processes preferably include a step for obtain a racemate by racemizing the optically active (R)-benzylamine derivative having a structure represented by the above formula (4) yielded as a by-product in the optical resolution step, and the racemate obtained in the step to obtain the racemate is used as a benzylamine derivative in the optical resolution step. Furthermore, in these processes, a ketone is preferably used as a solvent for the solution in the optical resolution step. The ketone is preferably acetone or methyl ethyl ketone.

Yet another aspect of the present invention provides a process for production of (1R,2S)-2-amino-1-(4-hydroxyphenyl)propan-1-ol, including the steps of optical resolution of a benzylamine derivative having a structure represented by the above formula (1) to obtain an optically active (S)-benzylamine derivative having a structure represented by the above formula (3), and performing a catalytic reduction of the optically active (S)-benzylamine derivative to obtain (1R,2S)-2-amino-1-(4-hydroxyphenyl)propan-1-ol.

### BEST MODE FOR CARRYING OUT THE INVENTION

A benzylamine derivative in an embodiment has a structure represented by the following formula (1):

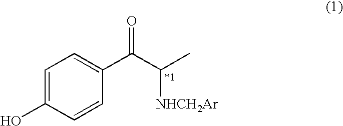

wherein Ar represents an aryl group that has 6 to 15 carbon atoms and may have a substituent, and *1 represents an asymmetric carbon atom.

An aryl group in the above formula (1) includes a phenyl group, a naphthyl group, and a biphenyl group. For this aryl group, a phenyl group is preferable because it is easily manufactured. When an aryl group has a substituent, examples of the substituent include halogen atoms (e.g. fluorine atom, chlorine atom, bromine atom and iodine atom), a nitro group, a nitroso group, a cyano group, an amino group, a hydroxyamino group, an alkylamino group having 1 to 12 carbon atoms, a dialkylamino group having 1 to 12 carbon atoms, an azide group, a trifluoromethyl group, a carboxyl group, an acyl group having 1 to 12 carbon atoms, an aroyl group having 7 to 12 carbon atoms, a hydroxyl group, an alkyloxy group having 1 to 12 carbon atoms, an aralkyloxy group having 7 to 12 carbon atoms, an aryloxy group having 6 to 12 carbon atoms, an acyloxy group having 1 to 12 carbon atoms, an aryloxy group having 7 to 12 carbon atoms, a silyloxy group having 3 to 12 carbon atoms, a sulfonyloxy group having 1 to 12 carbon atoms, and an alkylthio group having 1 to 12 carbon atoms. Among these substituents, it is preferable to use at least one selected from a hydroxyl group, an alkyloxy group having 1 to 12 carbon atoms, an aralkyloxy group having 7 to 12 carbon atoms, an acyloxy group having 1 to 12 carbon atoms, an aryloxy group having 7 to 12 carbon atoms, a silyloxy group having 3 to 12 carbon atoms, and a sulfonyloxy group having 1 to 12 carbon atoms. When the aryl group has a substituent, the number of the substituent is 1 to 3.

Among benzylamine derivatives having a structure represented by the above formula (1), a benzylamine derivative having a structure represented by the following formula (2) is preferable because it is easily manufactured. The benzylamine derivative having a structure represented by the following formula (2) is (R,S)-2-benzylamino-1-(4-hydroxyphenyl)propan-1-one:

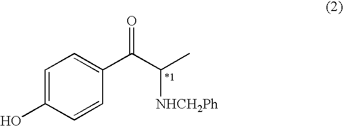

wherein Ph represents a phenyl group, and, and *1 represents an asymmetric carbon atom.

The benzylamine derivative having a structure represented by the above formula (1) is an optically inactive racemate, and for instance, it is obtained by a synthesis route where 4-hydroxypropiophenone is used as a starting material. Specifically, first, 4-hydroxypropiophenone is subjected to an addition reaction of bromine atoms, thereby 2-bromo-(4-hydroxyphenyl)propan-1-one is obtained. This addition reaction is, for example, represented by the following reaction formula (5):

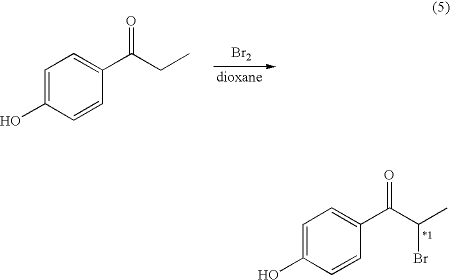

wherein *1 represents an asymmetric carbon atom.

For this addition reaction, methods described in Japanese Laid-Open Patent Publication Nos. 56-81560 and 60-188344 can be employed. In the method (A) described in the Japanese Laid-Open Patent Publication No. 56-81560, by adding drops of bromine to a solution of 4-hydroxypropiophenone, the second position of propan-1-one constituting 4-hydroxypropiophenone is brominated, while bromination of an aromatic ring is suppressed. In this bromination, for example, the solvent such as methanol, ethanol, and ethers is used. Examples of ethers include lower aliphatic acid ether and cyclic ether. Examples of lower aliphatic acid ether include ethyl ether, and n-butyl ether. Examples of cyclic ether include tetrahydrofuran and dioxane. In the method (B) described in the Japanese Laid-Open Patent Publication No. 60-188344, by using copper (II) bromide, the second position of propan-1-one constituting 4-hydroxypropiophenone is brominated, while bromination of an aromatic ring is suppressed. In this bromination using copper (II) bromide, as a solvent, chloroform, ethyl acetate, dioxane, N,N-dimethylformamide, and alcohols are used, ethyl acetate is preferably used, and a mixed solution of ethyl acetate and chloroform is more preferably used. In this method (B), since copper (I) bromide is remained even after the addition reaction of bromine, it is necessary to eliminate the copper (I) bromide. Therefore, along with production of 2-bromo-(4-hydroxyphenyl)propan-1-one, copper (I) bromide is discharged as a waste. In contrast, since a waste such as copper (I) bromide is not discharged, the method (A) is industrially superior. Accordingly, 2-bromo-(4-hydroxyphenyl)propan-1-one is preferably manufactured by the method (A).

Then, by a substitution reaction of 2-bromo-(4-hydroxyphenyl)propan-1-one obtained by this addition reaction, a benzylamine derivative having a structure represented by the above formula (1) is obtained. Specifically, in this substitution reaction, a bromine atom in 2-bromo-(4-hydroxyphenyl)propan-1-one is replaced with benzylamine in the presence of a base. This substitution reaction is represented, for example, by the following reaction formula (6):

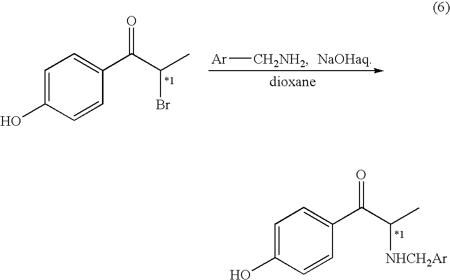

wherein Ar represents an aryl group that has 6 to 15 carbon atoms and may have a substituent, and *1 represents an asymmetric carbon atom.

A base used in this substitution reaction is not particularly limited, and specific examples include potassium hydroxide and sodium hydroxide. A solvent used in the substitution reaction may be methanol, ethanol, or ethers. The ethers include lower aliphatic acid ether and cyclic ether. The lower aliphatic acid ether includes ethyl ether and n-butyl ether. The cyclic ether includes tetrahydrofuran and dioxane. Among these solvents, ethers are preferable, and cyclic ethers are more preferable.

In the method for optical resolution of a benzylamine derivative in the embodiment is a method for optical resolution of a benzylamine derivative (racemate) having a structure represented by the above formula (1), and optically active mandelic acid can be used as an optical resolving agent. Examples of the optically active mandelic acid include (S)-mandelic acid and (R)-mandelic acid. In this method, the benzylamine derivative having a structure represented by the above formula (1) is optically resolved into an optically active (S)-benzylamine derivative having a structure represented by the following formula (3):

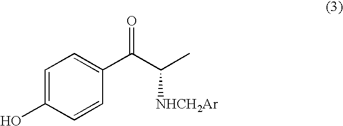

wherein Ar represents an aryl group that has 6 to 15 carbon atoms and may have a substituent,

and an optically active (R)-benzylamine derivative having a structure represented by the following formula (4):

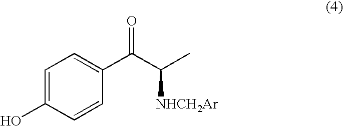

wherein Ar represents an aryl group that has 6 to 15 carbon atoms and may have a substituent.

This optical resolution method utilizes the fact that a relationship of diastereomer is established between optically active mandelic acid salt of the optically active (S)-benzylamine derivative having a structure represented by the above formula (3) and optically active mandelic acid salt of the optically active (R)-benzylamine derivative having a structure represented by the above formula (4). That is, (S)-mandelic acid salt of the optically active (S)-benzylamine derivative and (S)-mandelic acid salt of the optically active (R)-benzylamine derivative are in the relationship of diastereomer. In the same way, (R)-mandelic acid salt of the optically active (S)-benzylamine derivative and (R)-mandelic acid salt of the optically active (R)-benzylamine derivative are in the relationship of diastereomer. Such a pair of salts in the relationship of diastereomer each has different solubility to a solvent. That is, for a solvent in which the benzylamine derivative having a structure represented by the formula (1) is dissolved, (S)-mandelic acid salt of the optically active (S)-benzylamine derivative is insoluble, however, (S)-mandelic acid salt of the optically active (R)-benzylamine derivative is soluble. Furthermore, while (R)-mandelic acid salt of the optically active (S)-benzylamine derivative is soluble in this solvent, (R)-mandelic acid salt of the optically active (R)-benzylamine derivative is insoluble in the solvent. By utilizing such a difference in solubility between a pair of salts, the optically active (S)-benzylamine derivative and the optically active (R)-benzylamine derivative can be optically resolved in a solution containing a benzylamine derivative that is a racemate and optically active mandelic acid as an optical resolving agent.

In a production method of an optically active benzylamine derivative in the embodiment, the optically active (S)-benzylamine derivative having a structure represented by the above formula (3) is produced. This production method includes a step of optically resolving a benzylamine derivative having a structure represented by the above formula (1). In this optical resolution step, from a solution containing the benzylamine derivative having a structure represented by the above formula (1) and (S)-mandelic acid as an optical resolving agent, the optically active (S)-benzylamine derivative having a structure represented by the above formula (3) is precipitated as its (S)-mandelic acid salt. In the same manner as the above described optical resolution method, this optical resolution step utilizes the fact that a relationship of diastereomer is established between respective (S)-mandelic acid salts of the optically active (S)-benzylamine derivative having a structure represented by the above formula (3) and the optically active (R)-benzylamine derivative having a structure represented by the above formula (4).

In the optical resolution step, the amount of (S)-mandelic acid is preferably 1 mol or more based on the benzylamine derivative having a structure represented by the formula (1), more preferably 1 to 2 mol, and more preferably 1 to 1.5 mol. When the amount of (S)-mandelic acid is 1 mol or more, a yield of the optically active (S)-benzylamine derivative having a structure represented by the formula (3) is maximized.

An organic solvent may be used as a solvent in the optical resolution step, that is, a solvent dissolving the benzylamine derivative having a structure represented by the above formula (1). The organic solvent includes, for example, ketones and esters. Among these, from the viewpoint that an optical purity of an optically active benzylamine derivative to be obtained can be increased, ketones are preferable. Examples of the ketones include acetone, methyl ethyl ketone and methyl isobutyl ketone. Among these, from the viewpoint that the optical purity can be further increased, acetone or methyl ethyl ketone is preferable. A mixed solution of an organic solvent and water can be also used as a solvent. When the mixed solvent is used, a content of water in the mixed solvent is preferably 40 vol % or less.

The amount of the benzylamine derivative having a structure represented by the above formula (1) in a solvent is preferably 0.5 to 0.8 mmol/mL, and more preferably 0.5 to 0.6 mmol/mL. When this amount is 0.5 to 0.8 mmol/mL, the solubility of the benzylamine derivative is favorable, and a sufficient yield of the optically active (S)-benzylamine derivative is ensured.

When the benzylamine derivative and (S)-mandelic acid are dissolved in the solvent, it is preferable that the solvent is stirred in the state of being heated to its boiling point and refluxed. Thereby, a dissolution time of the benzylamine derivative and (S)-mandelic acid is shortened. The dissolution time is preferably 5 to 120 minutes, and more preferably 10 to 60 minutes.

When a salt of the optically active (S)-benzylamine derivative is precipitated, the solution in which the benzylamine derivative and (S)-mandelic acid are dissolved is subjected to a cooling treatment or a concentration treatment. From the viewpoint that an optical purity of the optically active (S)-benzylamine derivative is increased, it is preferable that the solution is subjected to at least a cooling treatment. The temperature of the solution in the cooling treatment is preferably 5 to 40° C., and more preferably 10 to 30° C. from the viewpoint that a yield and an optical purity of the optically active (S)-benzylamine derivative are increased. A time for the cooling treatment is preferably 10 to 300 minutes, and more preferably 30 to 200 minutes from the viewpoint that a yield and an optical purity of the optically active (S)-benzylamine derivative are increased.

A salt of the optically active (S)-benzylamine derivative thus obtained is, for example, washed and dried, according to need. Then, the salt of the optically active (S)-benzylamine derivative is treated with an acid and a base, thereby the optically active (S)-benzylamine derivative that is the target is obtained. Examples of the acid include hydrochloric acid. Examples of the base include an aqueous sodium hydroxide solution.

On the other hand, in this optical resolution step, (S)-mandelic acid salt of the optically active (R)-benzylamine derivative remains in the solution. This solution is treated with an acid and a base, thereby the optically active (R)-benzylamine derivative having a structure represented by the formula (4) is obtained. This optically active (R)-benzylamine derivative is produced as a by-product at the time of production of the optically active (S)-benzylamine derivative having a structure represented by the formula (3). The production method of the embodiment includes a step of racemizing the optically active (R)-benzylamine derivative to obtain a racemate. The racemate obtained in this step is used again as a benzylamine derivative that is used as a raw material in the optical resolution step.

When the optically active (S)-benzylamine derivative is produced from the benzylamine derivative as a raw material in the optical resolution step, a theoretical yield of the optically active (S)-benzylamine derivative cannot be 50% or more. In contrast, the production method of the present embodiment includes a step to obtain a racemate, and the racemate obtained from the optically active (R)-benzylamine derivative is reused as a raw material, therefore, a yield of the produced optically active (S)-benzylamine derivative of 50% or more is realized.

In the step to obtain a racemate, (S)-mandelic acid salt of the optically active (R)-benzylamine derivative is heated and stirred under a basic condition, thereby, a benzylamine derivative having a structure represented by the formula (1), that is, a racemate is obtained. A pH showing the basic condition in the step to obtain a racemate is preferably 13 or more from the viewpoint that a complete racemate is easily obtained. In other words, the racemization is carried out under a basic condition with a pH of 13 or more, thereby the reaction easily proceeds, and as a result, the reaction time is shortened, and the yield of the racemate is increased. In the step to obtain a racemate, a base for allowing pH to exhibit basicity is not particularly limited, and specific examples include sodium hydroxide and potassium hydroxide. A solvent used in the step to obtain a racemate is preferably a mixed solvent of water and an alcohol. The solvent in the step to obtain a racemate is preferably stirred in the state of being heated to its boiling point and refluxed. The solution containing the racemate thus obtained is subjected to a neutralization treatment with an acid such as hydrochloric acid, thereby the racemate is obtained as a crystal. The crystal of the racemate is washed and dried according to need, and then, it is used as a benzylamine derivative in the optical resolution step. When the optically active benzylamine derivative is produced, the step to obtain a racemate may be omitted.

The optically active (S)-benzylamine derivative having a structure represented by the above formula (3) obtained in the production method in the present embodiment is utilized as a precursor of (1R,2S)-2-amino-1-(4-hydroxyphenyl)propan-1-ol having a structure represented by the following formula (7), for example.

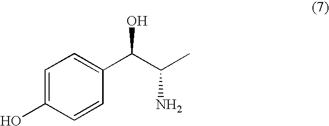

Specifically, a catalytic reductive reaction of the optically active (S)-benzylamine derivative provides (1R,2S)-2-amino-1-(4-hydroxyphenyl)propan-1-ol having a structure represented by the above formula (7). By this catalytic reductive reaction, the optically active (S)-benzylamine derivative is reduced with hydrogen in the presence of a catalyst, and the catalytic reductive reaction is represented by the following reaction formula (8):

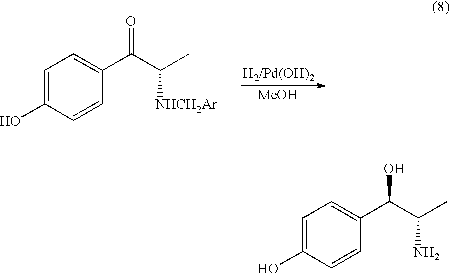

wherein Ar represents an aryl group that has 6 to 15 carbon atoms and may have a substituent.

Thus obtained (1R,2S)-2-amino-1-(4-hydroxyphenyl)propan-1-ol is broadly used as an optically active substance useful for a pharmaceutical intermediate.

Advantages obtained by the present embodiment will be described in the following.

A benzylamine derivative having a structure represented by the above formula (1) is significantly useful as a precursor of an optically active (S)-benzylamine derivative having a structure represented by the above formula (3). Among benzylamine derivatives having a structure represented by the above formula (1), a benzylamine derivative having a structure represented by the above formula (2) has a great deal of potential in industry since production thereof is easy.

In the optical resolution method of a benzylamine derivative, an optically active mandelic acid is used as an optical resolving agent, and the benzylamine derivative that is a racemate can be optically resolved into an optically active (S)-benzylamine derivative having a structure represented by the above formula (3) and an optically active (R)-benzylamine derivative having a structure represented by the above formula (4). Therefore, not only the optically active (S)-benzylamine derivative having a structure represented by the above formula (3), but also the optically active (R)-benzylamine derivative having a structure represented by the above formula (4) is easily obtained, and both of these optically active substances can be utilized for an application such as a pharmaceutical intermediate.

In the production method of an optically active benzylamine derivative, by the optical resolution step in which (S)-mandelic acid is used as an optical resolving agent, the optically active (S)-benzylamine derivative having a structure represented by the above formula (3) is easily obtained from a benzylamine derivative having a structure represented by the above formula (1) as a precursor. Further, the obtained optically active (S)-benzylamine derivative can be used as a precursor of (1R,2S)-2-amino-1-(4-hydroxyphenyl)propan-1-ol having a structure represented by the above formula (7). This (1R,2S)-2-amino-1-(4-hydroxyphenyl)propan-1-ol is an optically active substance useful as a pharmaceutical intermediate, therefore, the optically active (S)-benzylamine derivative has a great deal of potential in industry.

The above described production method preferably includes a step to obtain a racemate in addition to the optical resolution step, and the racemate obtained in the step to obtain a racemate is used as a benzylamine derivative that is a raw material in the optical resolution step. That is, according to this method, the optically active (R)-benzylamine derivative to be a by-product in the optical resolution step is reused as a raw material in the optical resolution step through the step to obtain a racemate. Therefore, in the optical resolution step where the racemate obtained in the step to obtain a racemate is used as a raw material, the benzylamine derivative in an amount to satisfy the shortage of the racemate may be newly provided as a raw material. That is, according to the production method containing the step to obtain a racemate, the amount in use of the benzylamine derivative as a new raw material is reduced. As a result, the yield of the optically active (S)-benzylamine derivative is increased. Without the production method described above, it cannot be possible to produce the optically active (S)-benzylamine derivative from the benzylamine derivative at a yield exceeding 50%. Furthermore, by repeating such a production method, the optically active (S)-benzylamine derivative is produced from the benzylamine derivative at a yield close to 100%, and thus, the production method including the step to obtain a racemate is significantly advantageous in the industrial viewpoint.

Among many kinds of organic solvents, ketones sufficiently ensure a dissolution gap between (S)-mandelic acid salt of the optically active (S)-benzylamine derivative and (S)-mandelic acid salt of the optically active (R)-benzylamine derivative. Therefore, it can be avoided that a salt of the optically active (S)-benzylamine derivative is precipitated, and at the same time, a salt of the optically active (R)-benzylamine derivative is precipitated. As a result, using ketones as a solvent for the solution in the optical resolution step enables an optical purity of the obtained optically active (S)-benzylamine derivative to be increased. Specifically, using acetone or methyl ethyl ketone as ketones enables an optical purity of the obtained optically active benzylamine derivative to be further increased.

The above described embodiment may be modified as shown below.

(S)-mandelic acid in the optical resolution step may be changed to (R)-mandelic acid. In this case, from a solution containing the benzylamine derivative having a structure represented by the above formula (1) and (R)-mandelic acid as an optical resolving agent, (R)-mandelic acid salt of the optically active (R)-benzylamine derivative having a structure represented by the above formula (4) is precipitated. In this way, the benzylamine derivative having a structure represented by the above formula (1) is optically resolved into the optically active (S)-benzylamine derivative having a structure represented by the above formula (3) and the optically active (R)-benzylamine derivative having a structure represented by the above formula (4). In this optical resolution step, the optically active (S)-benzylamine derivative having a structure represented by the above formula (3) is obtained in the state of being dissolved in the solvent. Therefore, when this optically active (S)-benzylamine derivative is reacted further in the post step, the optically active (S)-benzylamine derivative can be provided in the post step in the state of a solution. Accordingly, since the procedure of dissolving the optically active (S)-benzylamine derivative in the post step can be omitted, the optically active (S)-benzylamine derivative is obtained in the state of being highly advantageous to convenience for using in the post step.

### Examples

### Production of Benzylamine Derivative

To a solution (86 ml) of 43.0 g of 4-hydroxypropiophenone (286.3 mmol) in dioxane, 50.2 g (1.1 equivalence) of bromine was added at 30° C. or less, and the mixture was stirred for 5 minutes. This solution was heated to 90° C., hydrogen bromide in the solution was completely dispelled, and then, the solution was cooled to 20° C. or less (addition reaction represented by the reaction formula (5)). To the resultant solution, 31.2 g (1.0 equivalence) of benzylamine and 30 mL of an aqueous 40% sodium hydroxide solution were added dropwise, and the reaction mixture was stirred for 3 hours (substitution reaction represented by the reaction formula (6)). After the aqueous layer was removed, 65 mL of an isopropyl alcohol was added thereto, and the crystal filtered. Further, this crystal was washed with 65 mL of an isopropyl alcohol to obtain 2-benzylamino-1-(4-hydroxyphenyl)propan-1-one represented by the formula (2) as a white crystal (40.2 g, isolation yield of 55%).

The obtained white crystal was identified by ¹H-NMR. The result is shown in the following.

¹H-NMR (DMSO, 400 MHz/ppm) 1.19 (d, 3H), 3.55 (d, 1H), 3.67 (d, 1H), 4.28 (q, 1H), 6.84 (d, 2H), 7.23 (m, 1H), 7.29 (d, 4H), 7.86 (d, 2H), 10.4 (brs, 1H)<

### <Production of Optically Active Benzylamine Derivative>

### (Optical Resolution Step 1)

To a solution (750 mL) of 230 g of 2-benzylamino-1-(4-hydroxyphenyl)propan-1-one (900.9 mmol) in 90% acetone/10% water obtained in the above described “Production of Benzylamine Derivative”, 164.1 g (1.2 equivalence) of (S)-mandelic acid was added, and the mixture was stirred for 30 minutes in the state where the solution was refluxed. The solution was cooled to 20° C., and then stirred at the same temperature for 2 hours, thereby a crystal was precipitated. Then, by filtering the solution in which the crystal was precipitated, the crystal was separated. Further, the crystal was washed with a mixed solution (255 mL) of 90% acetone/10% water to obtain (S)-mandelic acid salt of (S)-2-benzylamino-1-(4-hydroxyphenyl)propan-1-one as a white crystal (174.7 g, isolation yield of 47.6% (based on the racemate), optical purity of 99.5% ee). Repetition of the above described procedure gave a predetermined amount of (S)-mandelic acid salt of (S)-2-benzylamino-1-(4-hydroxyphenyl)propan-1-one. The washing solution with which the filtrate and the crystal obtained by filtration were washed was collected as the recovery solution.

195.9 g of an white crystal, which was (S)-mandelic acid salt of (S)-2-benzylamino-1-(4-hydroxyphenyl)propan-1-one (480.8 mmol, optical purity of 99.9% ee) was dissolved in a solution containing 1050 mL of waster, 54 g of concentrated hydrochloric acid, and 30 mL of methanol. The resultant solution was neutralized with 2 mol/L of an aqueous sodium hydroxide solution, thereby a crystal was precipitated, and then, the crystal was separated by filtering the solution. Further, the crystal was washed with water (150 mL) to obtain (S)-2-benzylamino-1-(4-hydroxyphenyl)propan-1-one having a structure represented by the following formula (9) as a white crystal (121.5 g, isolation yield of 99.0%, optical purity of 99.5% ee):

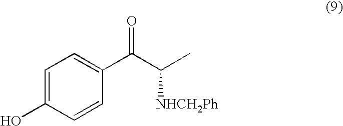

wherein Ph represents a phenyl group.

The obtained white crystal is identified by an optical purity (% ee) and a result of ¹H-NMR. The result of ¹H-NMR is shown in the following.

¹H-NMR (DMSO, 400 MHz/ppm) 1.19 (d, 3H), 3.55 (d, 1H), 3.67 (d, 1H), 4.28 (q, 1H), 6.84 (d, 2H), 7.23 (m, 1H), 7.29 (d, 4H), 7.86 (d, 2H), 10.4 (brs, 1H)

Optical purities (% ee) of (S)-mandelic acid salt of (S)-2-benzylamino-1-(4-hydroxyphenyl)propan-1-one and the optically active (S)-benzylamine derivative having a structure represented by the above formula (9) were calculated by analysis using optical resolution HPLC. While a sample portion of 10 mg out of the sample solution provided for the analysis by the optical resolution HPLC was dissolved in methanol, 1 mL of the solution measured in a measuring flask to be 10 mL was diluted so as to adjust to be 10 mL in a measuring flask by using the mobile phase as a diluting solvent. The analysis condition of optical resolution HPLC is shown in the following. Hereinafter, an “optical purity” descried in Examples indicates a value calculated according to this optical resolution HPLC.

Column: DAICEL CHIRALPAK (registered trademark) AD-H 4.6 mm×250 mm

Mobile phase: Hexane:IPA:Diethylamine 80:20:0.1

Column temperature: 40° C.

Flow rate: 0.5 mL/min

Detection wavelength: 254 nm

Injection amount: 10 μL

### (Step to Obtain Racemate)

The solvent of 1150 mL of the recovery solution collected in washing the filtrate and the crystal in the optical resolution step was distilled off, and then, the recovery solution was dried and solidified so as to obtain (S)-mandelic acid salt of (R)-2-benzylamino-1-(4-hydroxyphenyl)propan-1-one as a white crystal (195.9 g, isolation yield of 53.4% based on the racemate, optical purity of 69.4% ee).

186.1 g of the obtained white crystal (456.7 mmol, 69.4% ee) was dissolved in a dissolution solution containing 1040 mL of an aqueous 2 mol/L sodium hydroxide solution and 430 mL of methanol. The resultant solution was refluxed for 3 hours while stirring the solution, thereby a racemization reaction was carried out. The solution was neutralized with hydrochloric acid, thereby a crystal was precipitated, and then by filtering the solution, a crystal was separated. Further, the crystal was washed with water (160 mL) to obtain 2-benzylamino-1-(4-hydroxyphenyl)propan-1-one of the racemate as a white crystal (110.8 g, isolation yield of 48.2% based on the racemate in the “Optical resolution step 1”).

### (Optical Resolution Step 2)

The optical resolution step 2 was carried out in the same manner as in the above described the “Optical resolution step 1”, using the white crystal (racemate) obtained in the above described the “Step to obtain racemate”. As a result, (S)-2-benzylamino-1-(4-hydroxyphenyl)propan-1-one having a structure represented by the above formula (9) was obtained as a white crystal (isolation yield of 99.0%, optical purity of 99.5% ee). This white crystal was identified from an optical purity (% ee) and a result of ¹-NMR. According to the result, even by using the racemate obtained from the optically active (R)-benzylamine derivative as a raw material in the optical resolution step, it was confirmed that the optically active (S)-benzylamine derivative was obtained at a high yield. Therefore, it was proved that excellent advantages of increasing a yield of the optically active (S)-benzylamine derivative were obtained in the production process containing the step to obtain a racemate of, and it is found that the method is significantly advantageous in the industrial viewpoint.

1-4. (canceled)

5. A process for production of an optically active benzylamine derivative, wherein an optically active (S)-benzylamine derivative having a structure represented by the following formula (3) is produced from a benzylamine derivative having a structure represented by the following formula (1),
the process comprising:
a step of precipitating (S)-mandelic acid salt of the optically active (S)-benzylamine derivative from a solution containing the benzylamine derivative and (S)-mandelic acid as an optical resolving agent to optically resolve the benzylamine derivative; and
a step of racemizing, through heating under a basic condition, an optically active (R)-benzylamine derivative having a structure represented by the following formula (4) yielded as a by-product in the optical resolution step to obtain a racemate,
wherein the racemate obtained in the step to obtain the racemate is used as a benzylamine derivative in the optical resolution step, 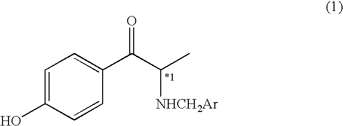
wherein Ar represents an aryl group that has 6 to 15 carbon atoms and may have a substituent, and *1 represents an asymmetric carbon atom, 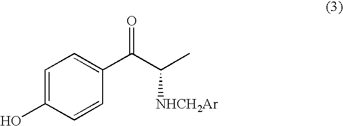
wherein Ar represents an aryl group that has 6 to 15 carbon atoms and may have a substituent, 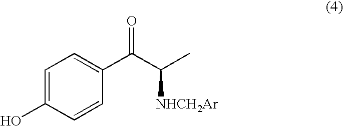
wherein Ar represents an aryl group that has 6 to 15 carbon atoms and may have a substituent.

6. A process for production of an optically active benzylamine derivative, wherein an optically active (S)-benzylamine derivative having a structure represented by the following formula (3) is produced from a benzylamine derivative having a structure represented by the following formula (1),
the process comprising:
a step of precipitating (R)-mandelic acid salt of an optically active (R)-benzylamine derivative having a structure represented by the following formula (4) from a solution containing the benzylamine derivative and (R)-mandelic acid as an optical resolving agent to optically resolve the benzylamine derivative; and
a step of racemizing, through heating under a basic condition, an optically active (R)-benzylamine derivative to obtain a racemate,
wherein the racemate obtained in the step to obtain the racemate is used as a benzylamine derivative in the optical resolution step, 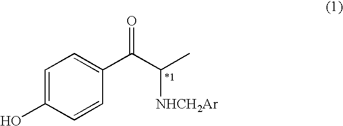
wherein Ar represents an aryl group that has 6 to 15 carbon atoms and may have a substituent, and *1 represents an asymmetric carbon atom, 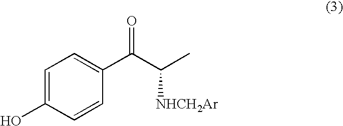
wherein Ar represents an aryl group that has 6 to 15 carbon atoms and may have a substituent, 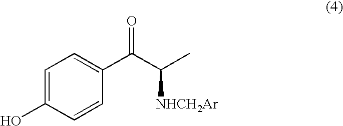
wherein Ar represents an aryl group that has 6 to 15 carbon atoms and may have a substituent.

7. (canceled)

8. The process for production of an optically active benzylamine derivative according to claim 5, wherein a ketone is used as a solvent for the solution in the optical resolution step.

9. The process for production of an optically active benzylamine derivative according to claim 8, wherein the ketone is acetone or methyl ethyl ketone.

10. (canceled)

11. The process for production of an optically active benzylamine derivative according to claim 5, wherein the racemization of the optically active (R)-benzylamine derivative in the step of obtaining the racemate is carried out by adding the optically active (R)-benzylamine derivative to a solvent containing aqueous sodium hydroxide solution and methanol, and, thereafter, heating the solution to its boiling point.

12. The process for production of an optically active benzylamine derivative according to claim 6, wherein a ketone is used as a solvent for the solution in the optical resolution step.

13. The process for production of an optically active benzylamine derivative according to claim 12, wherein the ketone is acetone or methyl ethyl ketone.

14. The process for production of an optically active benzylamine derivative according to claim 6, wherein the racemization of the optically active (R)-benzylamine derivative in the step of obtaining the racemate is carried out by adding the optically active (R)-benzylamine derivative to a solvent containing aqueous sodium hydroxide solution and methanol, and, thereafter, heating the solution to its boiling point.
